(19) United States
(12) Patent Application Publication
(10) Pub. No.: US 2004/0086559 A1
(43) Pub. Date: May 6, 2004

(54) Pharmaceutical tablet comprising paroextine mesylate

(76) Inventors: Theodorus Hendricus Antonius Peters, Arnhem (NL); Frans van Dalen, Boxtel (NL); Jacobus Maria Lemmens, Mook (NL); Frantisek Picha, Brno (CZ)

Correspondence Address: MARK R. BUSCHER, P.O. BOX 161, CATHARPIN, VA, 20143, US

(21) Appl. No.: 10/250,788

(22) PCT Filed: Dec. 20, 2001
(86) PCT No.: PCT/EP01/15115

(30) Foreign Application Priority Data
Jan. 11, 2001 (DE) 201 00 529.8

(51) Int. Cl. A61K 31/205; A61K 9/20

(52) U.S. Cl. 424/465; 514/554

(57) ABSTRACT

The invention is directed to novel and advantageous pharmaceutical compositions for oral administration of the antidepressant compound paroxetine. The composition is in the form of a tablet having a dissolution rate of at least 90% in 30 minutes measured with a paddle apparatus according to US Pharmacopoeia.

The present invention is directed to novel and advantageous compositions for oral administration of the antidepressant compound paroxetine.

Pharmaceutically active compounds with antidepressive properties are described in U.S. Pat. No. 4,007,196. An especially important compound among them is paroxetine, (−)trans-4-(4′-fluorophenyl)-3-(3′,4′-methylenedioxyphenoxymethyl)piperidine, having the formula

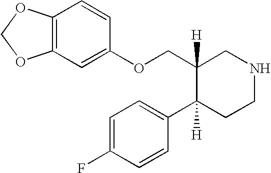

Paroxetine is currently used in medicinal practice for the preparation of a medicament having selective serotonine reuptake inhibition activity and is useful for the management of the various kinds of depressions. In the marketed medicament, such as in tablets for oral administration sold under the brand names Paxil® or Seroxat®, paroxetine is present in the form of the hydrochloride hemihydrate. Paroxetine hydrochloride hemihydrate is disclosed in EP 223403; it is a stable crystalline acid addition salt of paroxetine suitable for the preparation of oral dosage forms of paroxetine, such as tablets.

Another acid addition salt of paroxetine suitable for manufacturing tablet dosage forms for oral administration is paroxetine methane sulfonate (mesylate). This compound has been disclosed in WO 98/56787. In several aspects, this salt has better properties than the currently marketed paroxetine hydrochloride hemihydrate.

Several pharmaceutical compositions for oral administration of paroxetine mesylate were suggested in the prior art. A known tablet composition comprising paroxetine mesylate is disclosed in DE 199 18 588. The composition according to this prior art document further comprises dicalcium phosphate dihydrate as diluent, sodium starch glycollate as disintegrant and magnesium stearate as lubricant. Nothing is known about parameters of the tablet prepared from such composition and their pharmaceutical usefulness (stability etc.).

Other experimental tablet compositions of paroxetine mesylate are disclosed in CH 690 024. In this prior art document it is discussed that the dissolution rates of the compositions known from DE 199 18 588 are very poor (27% after 15 minutes and 59% after 30 minutes, respectively, when measured according to a paddle apparatus of US Pharmacopoeia (USP) using 0.1 M hydrochloric acid). According to CH '024 the dissolution rate of paroxetine mesylate containing compositions can be significantly improved by using water soluble diluents based on carbohydrates instead of dibasic calcium phosphate dihydrate. Physical parameters and stability of the prepared tablets are not reported.

The compositions known from prior art represent only experimental compositions which were not subjected to necessary testing which would serve as a basis for approval of their actual applicability in medicine. Consequently, it is not known up to now how an approvable single dose unit of paroxetine mesylate for oral application should look like, what should be the mass and size of such dosage unit, what should be the basic physical parameters thereof and how the dosage units should be conveniently packed and delivered for immediate use by a patient. None of the disclosed compositions were shown to comply with requirements of authorities approving pharmaceutical products for marketing and use.

A further problem of the previously known compositions is their bitter taste caused by the active ingredient paroxetine mesylate.

It is an object of the present invention to provide pharmaceutical dosage units for peroral administration of paroxetine in a form of tablets comprising paroxetine mesylate as the active ingredient, having improved dissolution rates and which do not comprise a water soluble diluent.

It is a further object of the present invention to provide paroxetine mesylate containing pharmaceutical dosage forms having an acceptable taste.

These objects are attained by the compositions and uses defined in the independent claims.

The dependent claims define preferred embodiments of the present invention.

We have surprisingly found that the dissolution rate of paroxetine mesylate containing compositions can be improved even in the absence of water soluble, carbohydrate based diluents by the use of anhydrous calcium hydrogen phosphate as diluent. Furthermore, we have found that the bitter taste of paroxetine mesylate is masked by anhydrous calcium hydrogen phosphate.

The compositions according to the invention comprise 1 to 200 mg paroxetine mesylate (calculated on the basis of the free base), preferably 5 to 100 mg, most preferred 10 to 50 mg, e.g. 10, 20, 30, 40 or 50 mg per single dosage unit. The active ingredient paroxetine mesylate may be applied in a crystalline or non-crystalline form, as described in WO 98/56787.

The compositions further comprise anhydrous calcium hydrogen phosphate which improves the dissolution rate of the compositions according to the invention and also masks the bitter taste of the active ingredient. Anhydrous calcium phosphate is present in an amount of 70 wt.-% to 90 wt.-%, preferably in the range of 80 to 88 wt.-%, most preferred in an amount of at least 86 wt.-%.

The compositions of the invention may further comprise at least one disintegrant such as alginic acid, starch, sodium lauryl sulfate, colloidal silicon dioxide, crospovidon or sodium starch glycollate, the latter being most preferred. The disintegrant is present in an amount from 0.5 wt.-% to 5 wt.-%, preferably in the range of 1 to 2 wt.-%.

Commonly used lubricants and/or glidants may also be included in the compositions of the invention. Preferably, the compositions in accordance with the present invention comprise magnesium stearate as lubricant in an amount of from 1 to 3 wt.-%.

Other commonly used auxiliary agents such as colourants, binders or surface active agents may also be included in the compositions.

According to a most preferred embodiment of the invention, the composition is formulated as a tablet comprising a core containing the active ingredient as well as a film coating. Any commonly used film coating material may be used in accordance with the invention, the use of water-dispersible coating materials, e.g. hydroxypropylmethylcellulose, hydroxypropylcellulose or mixtures thereof, being preferred.

For administration to humans in the management of various forms of depressions and related disorders curable by paroxetine, necessary daily dosages of paroxetine mesylate for an average adult patient should generally be in the range of from 5 to 80 mg of paroxetine. Thus, individual tablets of the present invention are designed in such way to fulfill most convenient dosage regimens by a single dose administration. In practice, the physician will determine the actual dosage which will be the most suitable for an individual patient, based on his/her age and individual response. The dosage forms of the present invention are believed to cover most of the average cases but there can, of course, be individual instances where higher or lower dosages are required. Thus, tablets according to the present invention are preferably scored to gain a possibility to be divided into at least two subunits, or several tablets may be ingested at once. The size, shape and mass of the tablets of the present invention is selected in such manner that the tablet can be conveniently swallowed by a patient. The tablet has preferably a round shape and a diameter not more than 13 mm (e.g. 9 mm at tablets comprising paroxetine mesylate equivalent to 20 mg of paroxetine). Alternatively, an oval shape of the tablet is also suitable. Having a relative content of paroxetine mesylate 6.5-7.3%, the total mass of a tablet of e.g. 20 mg strength is preferably about 348.7-385.5 mg. However, the tablets of the present invention are not limited to aforementioned relative content of paroxetine mesylate.

For the preparation of a commercially applicable tablet according to the present invention a suitable solid tabletting mixture comprising paroxetine mesylate and suitable inactive excipients is first to be prepared in bulk. Such mixture may be prepared by several methods.

A first suitable method (a wet granulation method) comprises granulating paroxetine mesylate, an inert diluent and/or other auxiliary substances by the aid of a granulating solvent (e.g. water) to form wet granules which are then dried and sieved to obtain a granulate. The pre-blend granulate is mixed thereafter with a disintegrant (advantageously in amounts of between 1-2% of the total mass) and subsequently with a lubricant (advantageously in amounts of between 1.5-2% of the total mass) to form a final granulate which is then screened to obtain the tabletting mixture.

A second method (a dry compaction method) comprises a granulation procedure wherein no granulating solvent is used in preparation of the pre-blend granulate.

A third method (a direct compression method) comprises one-step mixing of the active substance and inactive ingredients in solid state to provide a homogeneous mixture, and compression of the mixture into tablets.

The tablet prepared by any of the above methods represents a tablet core of the final product; preferably the surface of the tablet core is subsequently coated by a film-coat. In any of the production methods, care has to be taken to select excipients and process conditions which allow the production of tablets with desired content and mass uniformity and with proper physical parameters. Moreover, selection of excipients and tabletting conditions should be focused also to provide a tablet with a desired chemical and physical stability. In particular, undesirable reactions between paroxetine mesylate and excipients, environmental moisture and oxygen should be prevented. The key aspects for successful production of tablets of desired quality and stability are e.g. the nature of excipients (e.g. chemical reactivity or hygroscopicity thereof), microenvironmental pH, content of residual water in tablet mixture, particle size of the active substance etc.

The cores of the tablets according to the present invention may be produced by any of the above methods. Based on preformulation studies, the wet granulation method is advantageous as it provides desired weight and content uniformity of the produced tablets. As far as excipients are concerned, the preferred inert diluent is anhydrous calcium hydrogen phosphate, the preferred disintegrant is sodium starch glycollate and the preferred lubricant is magnesium stearate. The preferred solvent for granulation is water.

Coating of the paroxetine mesylate tablets prepared by any of the above tabletting methods is an advantageous feature as it makes swallowing easier and also protects the tablet composition against physical and chemical damages. The coating of the tablets of the present invention may comprise any suitable coating agent having preferably water-dispersible character. Such agent allows rapid contact of the tablet core with stomach fluids and, accordingly, it has only low influence on the desired release rate of the active substance.

Coating may be performed by any conventional coating method, such as perforated pan coating, dry coating, fluid bed coating or the like. The coating material is dispersed in an aqueous solvent, sieved and applied onto the tablet cores. The coating process is stopped when the theoretical increase in tablet weight is reached. A pharmaceutically acceptable colouration agent may optionally be added to the coating material for proper visual characterization of the made or used tablet.

It is a specific feature of the invention that the tablet does not comprise an inert water soluble or hydrophilic diluent, particularly that of a carbohydrate structure, as they are specifically listed in CH 690024. As it will be shown below, the tablets not comprising such diluent/s still have desired release rates and moreover pass the pharmaceutically necessary criteria of physical and chemical stability.

The use of anhydrous calcium hydrogen phosphate as a solid diluent, particularly when processed by a wet granulation method, is advantageous, because calcium hydrogen phosphate can efficiently mask the bitter taste of paroxetine mesylate. In the tablets of the invention, particularly those comprising a relative content of paroxetine mesylate less than 7.3% of the total mass of the tablet and comprising anhydrous calcium hydrogen phosphate in amounts at least 86.0% of the total mass of a tablet, no bitter taste has been observed at organoleptic investigation. This has not been disclosed in prior art.

The tablets according to the present invention meet the general criteria and requirements of European and U.S. Pharmacopoeias and they comply with all requirements stated in legal provisions regulating production and marketing of pharmaceutical products. The tablets can be reliably produced in industrial scale.

Proper hardness of a tablet assures safety and economy in handling with the produced tablet core in subsequent steps of coating and packing. We have found that a hardness not less than 30 N, as measured by a standard pharmaceutical method (e.g. Method 2.9.8 of European Pharmacopoeia), represents a proper lower limit in this respect for tablets comprising paroxetine mesylate.

The selection of excipients and coating also influences the overall release rate of paroxetine mesylate from the tablet matrix after ingestion. The release rate has subsequently an influence on plasma concentrations of paroxetine.

Taking into consideration the plasma concentration of paroxetine obtainable after administration of commercially available tablets of Seroxat® or Paxil®, we have proven that a paroxetine mesylate tablet which complies with the limits of hereinafter specified disintegration and dissolution tests provides—at the same dosage—essentially similar rate and extent of absorption of paroxetine in human body fluids as the marketed tablets having paroxetine hydrochloride as the active ingredient.

The tablets of the present invention exhibit a disintegration time of less than 15 minutes. The disintegration test is performed by a standardized pharmacopoeial method, e.g. by method 2.9.1 of European Pharmacopoeia or by method <701> of U.S. Pharmacopoeia (both methods are essentially equivalent).

The tablets of the invention exhibit a dissolution profile characterized in that at least 75% of the paroxetine content is liberated from the tablet matrix in 30 minutes, whenever determined in 0.1 M hydrochloric acid or in a simulated gastric fluid by a standardized pharmacopoeial method e.g. as specified in Method <711> of U.S. Pharmacopoeia, using a paddle apparatus of a preselected speed, e.g. from 50 to 100 rpm. The content of paroxetine in dissolution medium may be determined by any common technique which proves sufficient selectivity and accuracy. UV-spectrometry and high performance liquid chromatography are examples of such techniques.

According to a preferred embodiment, the tablets of the invention exhibit a dissolution profile characterized in that at least 90% of the paroxetine content is liberated in 30 minutes under the same conditions of measurement as specified above. It was surprisingly found that the tablet composition does not require the presence of water soluble or hydrophilic diluents, particularly those based on carbohydrates, in order to reach such dissolution profile, contrary to the disclosure of CH 690024. For example, tablets comprising anhydrous calcium hydrogen phosphate as the only solid diluent, less than 2% of sodium starch glycollate as a disintegrant and less than 2% of magnesium stearate as a lubricant, particularly those having a content of paroxetine mesylate between 6.5-7.3% of the total mass of the tablet, hardness of the tablet not less than 30 N and disintegration time not longer than 15 minutes, exhibit a dissolution rate from 91 to 98% in 30 minutes, if measured by the US Pharmacopoeia paddle apparatus in 0.1 M HCl or in simulated gastric fluid at a paddle speed of between 60 and 80 rpm.

The tablets of the present invention provide mean peak plasma levels of paroxetine in an average adult patient in between about 5 to about 7 hours after single dose oral administration. This concentration profile assures proper therapeutic response after administration by a patient, i.e. a response comparable to that of the commercially available tablets comprising paroxetine hydrochloride of equivalent strengths.

The applicability of tablets of the present invention in a therapy of humans has been proven by several pharmacokinetic studies, with a sufficient number of healthy human volunteers (both male and female) and in comparison with the commercially available tablets comprising paroxetine hydrochloride as the active ingredient. The studies were carried out in accordance with principles of Good Clinical Practice (GCP) and the Declaration of Helsinki. GCP requirements provide a legal standard under which the results of testing on humans are considered validated and may be used as a basis for evaluation of a particular drug within the process for obtaining marketing authorization.

The pharmacokinetic characteristics of paroxetine mesylate containing tablets of the invention were determined by statistical analysis of the pharmacokinetic parameters obtained from the found values of blood plasma levels of paroxetine after ingestion of a tablet, in a randomized double-blind cross-over fashion. The analyzed parameters comprised the value of the maximum concentration of paroxetine in a blood sample [cₘₐₓ, in ng/ml], the time in which such peak concentration has been reached [tₘₐₓ, in hrs], and the integral concentration of paroxetine in blood [AUC]). The analytical method used for the determination of paroxetine concentrations in plasma—high performance liquid chromatography combined with double mass spectroscopy (LC/MS/MS method)—was sufficiently validated according to current standards.

All pharmacokinetic parameters of paroxetine mesylate tablets according to the present invention (tₘₐₓ, cₘₐₓ, AUC) passed the requirements for bioequivalence with commercially available tablets comprising paroxetine hydrochloride hemihydrate as the active substance.

A commercially applicable tablet should exhibit sufficient stability during storage; stability parameters of any tablet comprising paroxetine mesylate as the active ingredient have not been disclosed in the prior art.

According to a preferred embodiment of the invention, a film-coated tablet of paroxetine mesylate having a pharmaceutically acceptable stability for at least 36 months if stored at 25° C. and 60% relative humidity is provided. Such stability allows handling of the tablet under ambient conditions without the need of specific protective means and is long enough to assure necessary safety of the commercial product during its manufacture, storing, transport and use of the product both at the manufacturer, distributor, pharmacist and final user.

Commercially useful tablets should be packed in an appropriate primary package for easy handling, distribution and storage and for assurance of proper stability of the tablet at prolonged contact with the environment during storage. Based on extensive stability studies performed, we have found that a convenient means or article for providing/packing tablets according to the present invention is a package type wherein the tablets are packed separately from each other in a single unit conventionally called blister or strip. Blisters/strips may be made by techniques known in the art from a support and a top foil made from pharmaceutically acceptable materials. One single blister/strip may advantageously bear 5, 10, 14, 20, or 28 tablets.

Such type of package is advantageous also with regard to handling, allows easy control of amounts of spent tablets and protects against abuse.

A suitable package for tablets of the present utility model is e.g. a PVC/PE/PVDC/Al blister or an Al/Al strip. 10 or 20 film-coated tablets may be packaged in blank blisters/strips, 14 or 28 tablets may be packaged both in blank or in calendar packs. 50 tablets may be also available via a “SUD” blister (single unit delivery pack for hospitals) comprising 5 tablets per blister.

The PVC/PE/PVDC/Al blister preferably comprises a top composite foil of 250 μm thick PVC foil, 25 μm thick PE foil, with a PVDC coating welded on an internally film-coated 20 μm aluminium semirigid support. This thermally weldable film prevents film-coated tablets from a direct contact with the metal while allowing strips to weld on the support. Each tablet is individually sealed in its pocket and thereby protected from shocks and from microbial and chemical contamination.

The Al/Al strip preferably comprises an internally film-coated 45 μm thick top aluminum foil welded on an internally film-coated 20 μm aluminum semirigid support.

For delivery and transport, one or several blisters/strips are packaged in a lithographed carton box and each box contains an insert leaflet with prescribed instructions for use. An alternative suitable package for the tablets according to the invention is a bottle made from high density polyethylene (HDPE).

Tablets in accordance with the present invention are useful in treatment of various diseases such as various forms of depression, anxiety, obsessive compulsive disorder, post-traumatic stress disorder, panic disorder, social phobia, alcoholism, migraine, anorexia, bulimia, pre-menstrual tension syndrome, tobacco withdrawal syndrome or chronic pain.

The following examples further illustrate the invention.

### EXAMPLE 1

Paroxetine (as mesylate) 20 mg tablets.

a) Tablet Composition

| | |
|---|---|
| Composition of the tablet core: | |
| paroxetine mesylate | 25.83 mg |
| calcium hydrogenphosphate anhydrous | 317.75 mg |
| sodium starch glycollate | 5.95 mg |
| magnesium stearate | 7.00 mg |
| Composition of coating: | |
| hydroxypropylmethyl cellulose | 3.912 mg |
| hydroxypropylcellulose | 3.912 mg |
| colourants (E 171, 172) | 2.766 mg |

b) Tabletting Process (100.000 tablets)

Anhydrous Calcium hydrogenphosphate was transferred into a granulating device.

A 45%(w/w) solution of paroxetine mesylate in water was added to the mixture to form a granulate

The formed granules were dried at 40° C. in vacuum

The granulate was mixed with sodium starch glycollate and subsequently with magnesium stearate in a free fall mixer.

The formed granulate was sieved through a 1.0 mm sieve.

Tablet cores were made on a rotary press with a round biconvex two-sided scored punch of a diameter of 9 mm.

Tablets were film-coated on a comparable device. The coating material was dispersed in an aqueous solvent, sieved through a 0.8 mm sieve and sprayed onto the tablet cores. The coating process was stopped when the theoretical increase in tablet weight was reached.

c) Package Process

Bulk coated tablets were packed into a PVC/PE/PVDC/Al blister and into an Al/Al strip with a blister machine; welded foils bearing sealed tablets were cut into pieces bearing 10 tablets and finished blisters were packed into a lithographed carton box.

### EXAMPLE 2

Properties of a typical batch of paroxetine (as mesylate) 20 mg tablets

average weight: 368.5 mg

content of paroxetine (as a base): 19.7 mg

mass uniformity: complies with Ph.Eur. 2.9.5.

content uniformity: complies with USP <905>

hardness: 115 N

disintegration time: less than 5 minutes

dissolution time (0.1 N HCl, paddle, 80 rpm, UV detection): 97% in 30 minutes

impurities present: one identified impurity 0.2%, one unidentified impurity 0.1%, sum of all impurities 0.3%.

### EXAMPLE 3

Another batch of paroxetine (as mesylate) 20 mg tablets prepared as in Example 1 was subjected to a testing of dissolution using a paddle apparatus according to US Pharmacopoeia and the following conditions:

A) Simulated gastric fluid (pH 1.2), 60 rpm

B) 0.1 M HCl, 80 rpm.

C) 0.1 M HCl, 60 rpm

Concentration of dissolved paroxetine was measured by validated UV-spectrophotometric method.

The results are shown in the following table:

Dissolution in % after

| Method | 15 min | 30 min | 45 min | 60 min |
|---|---|---|---|---|
| A | 73 | 91 | 96 | 98 |
| B | 95 | 98 | 98 | 98 |
| C | 85 | 101 | 103 | 104 |

### EXAMPLE 4

Stability testing of paroxetine (as mesylate) 20 mg tablets

Bulk produced tablets having the composition as shown in Example 1 and the same tablets packed in the both types of blister packs of the same Example were placed into a thermostated chamber adjusted to 25±2° C. and 60±5% relative humidity and stored for two years. Samples were taken at t=0, 3, 6, 9, 12, 18, 24 and 36 months.

The samples were tested for the following parameters:

Hardness

Uniformity of mass/average mass

Uniformity of mass of broken tablets

Disintegration time

Water content

Dissolution

Content uniformity

Identification, assay, and purity of paroxetine

Identification of mesylate counter-ion

Microbial contamination

The tablets complied with the specification set forth in Examples 1 and 2 for the entire storage time of 36 months. No significant change in physical and chemical parameters of the produced tablets were observed.

### EXAMPLE 5

Testing of Bioequivalence

Paroxetine 20 mg (as mesylate) tablets were tested against Seroxat® in a single-dose randomized 2-way crossover bioequivalence study under fasted conditions. 48 healthy male and female volunteers were enrolled. Blood samples were taken during a 120 hour time interval. Plasma paroxetine levels were measured by a validated LC/MS/MS method. The study was carried out in accordance with the principles of Good Clinical Practice and the Declaration of Helsinki.

The pharmacokinetic parameters Cₘₐₓ, and AUC (normal and In-transformed) were subjected to the analysis of variance (ANOVA). The 90% conventional confidence intervals of the point estimate of the ratio of the test product relative to the reference product on the ln-transformed values were within the conventional bioequivalence limits of 80 and 125%. Both products were proven bioequivalent. Calculated mean values of tₘₐₓ after a single dose administration of paroxetine mesylate 20 mg tablets were 5.35 hrs and 5.85 hrs in two subsequent studies.

### EXAMPLE 6

Paroxetine Mesylate Tablets

Using compositions proportional to that as shown in Example 1 and using the same production and package process, tablets containing paroxetine mesylate equivalent to 10, 30, 40 and 50 mg of paroxetine were prepared as well.

1. A pharmaceutical composition in the form of a tablet comprising paroxetine methane sulfonate having a dissolution rate of at least 90% in 30 Minutes measured with a paddle apparatus according to US Pharmacopoeia, with the exception of compositions comprising a water soluble and/or hydrophilic diluent.

2. Composition according to claim 1, comprising paroxetine methane sulfonate in a amount equivalent to 10-50 mg of paroxetine base.

3. Composition according to claim 1 or 2, comprising a tablet core containing paroxetine methane sulfonate and anhydrous calcium hydrogen phosphate.

4. Composition according to any of claims 1-3, further comprising a film coating covering the tablet core.

5. Composition according to claim 4,
characterized in that the tablet core additionally comprises at least one disintegrant and/or at least one lubricant.

6. Composition according to claim 5,
characterized in that the disintegrant is sodium starch glycollate.

7. Composition according to claim 5,
characterized in that the lubricant is magnesium stearate.

8. Composition according to any of claims 4-7,
characterized in that the film coating comprises a mixture of hydroxypropylmethylcellulose and hydroxypropylcellulose.

9. Composition according to any of claims 4-8,
characterized in that the film coating comprises a colouring agent.

10. Composition according to any preceding claim,
characterized in that the paroxetine methane sulfonate is present in crystalline form.

11. Composition according to any preceding claim having a hardness of at least 30N.

12. Composition according to any preceding claim,
characterized in that it has no bitter taste.

13. Composition according to any preceding claim,
characterized in that it has a round shape and at least one scoring on at least one side.

14. Composition according to any preceding claim having a pharmaceutically acceptable stability for at least 6 months if stored at 40° C. and 75% relative humidity and/or for at least 36 months if stored at 25° C. and 60% relative humidity.

15. Composition according to any preceding claim having a disintegration time of less than 15 minutes.

16. Composition according to any preceding claim providing mean peak plasma levels of paroxetine in an average adult patient in 5-7 hours after a single dose oral administration.

17. Use of anhydrous calcium hydrogen phosphate for improving the dissolution rate of paroxetine methane sulfonate.

18. Use of calcium hydrogen phosphate for masking the bitter taste of paroxetine methane sulfonate.
